SEQUENCE LISTING (5 sequences)

SEQ ID NO: 1 - RNA, 4 nt, Artificial Sequence
  Synthesized
ncca

SEQ ID NO: 2 - RNA, 7 nt, Artificial Sequence
  Synthesized
gnnnnnu

SEQ ID NO: 3 - DNA, 18 bp, Artificial Sequence
  Synthesized
gcccgagacgcctcccga

SEQ ID NO: 4 - DNA, 29 bp, Artificial Sequence
  Synthesized
aaaaggtggtagcatccaatacctgtctc

SEQ ID NO: 5 - DNA, 29 bp, Artificial Sequence
  Synthesized
aatattggatgctaccaccttcctgtctc

(19) United States
(12) Patent Application Publication
(10) Pub. No.: US 2015/0030667 A1
(43) Pub. Date: Jan. 29, 2015

(54) COMPOSITIONS AND METHODS FOR TREATING UNDERACTIVE BLADDER

(71) Applicant: LIPELLA PHARMACEUTICALS, INC., Pittsburgh, PA (US)

(72) Inventors: Jonathan KAUFMAN, Pittsburgh, PA (US); Michael B. CHANCELLOR, Pittsburgh, PA (US)

(73) Assignee: LIPELLA PHARMACEUTICALS, INC., Pittsburgh, PA (US)

(21) Appl. No.: 14/225,154

(22) Filed: Mar. 25, 2014

Related U.S. Application Data
(60) Provisional application No. 61/805,114, filed on Mar. 25, 2013.

(51) Int. Cl. A61K 31/713; A61K 9/127; A61K 31/325; A61K 31/685

(52) U.S. Cl. CPC A61K 31/713; A61K 31/685; A61K 9/127; A61K 31/325
USPC 424/450; 514/114; 514/44 A; 514/490

(57) ABSTRACT

Methods and compositions for treating underactive bladder and/or symptoms thereof are disclosed, wherein the composition including an effective amount of a therapeutic agent and a pharmaceutically acceptable lipid vehicle is locally administered to the bladder, thereby treating the underactive bladder and/or symptoms thereof.

### CROSS-REFERENCE TO RELATED APPLICATIONS

This application claims priority to and the benefit of U.S. Provisional Application No. 61/805,114 entitled “Compositions and Methods for Treating Under Active Bladder” filed Mar. 25, 2013.

### BRIEF SUMMARY OF THE INVENTION

In an embodiment, an underactive bladder condition or symptoms thereof are treated by locally administering a composition comprising a pharmaceutically acceptable vehicle and an effective amount of a therapeutic agent.

In another embodiment, a composition for treating an underactive bladder condition or symptoms thereof is described. The composition comprises a pharmaceutically acceptable vehicle and an effective amount of a therapeutic agent.

### DESCRIPTION OF DRAWINGS

FIG. 1: The increased duration of voiding contraction in pink tracing of rats pre-treated by instillation of BoNT is considered an accepted index of efficacy for agents improving detrusor underactivity or UAB. Notice the difference in effect of neostigmine in saline vs. BoNT treated rats. BoNT treatment in this animal model mimics to an extent the condition of UAB.

FIGS. 2(A) and 2(B): Bar graphs from CMG of control female rats and groups previously treated with BoNT. Neostigmine infusion caused a significant increase in duration of contraction 2(B) of BoNT treated rats and also raised the amplitude of contraction 2(A) (n=5). Data support the hypothesis that inhibition of endogenously release ACh in bladder by direct enzymatic inhibition with neostigmine can blunt the diminishing effect of BoNT on bladder contractility, used here to mimic UAB condition.

FIG. 3(A): The confocal photomicrograph of the bladder 24 hrs after instillation of NGF antisense oligos tagged with a red fluorescent dye (TYE563) conjugated with LP to demonstrate bladder UT uptake of fluorescent labeled oligos.

FIGS. 3(B) and 3(C) Effect of delivered antisense inhibitory oligonucleotide on NGF expression in the urothelium was studied by green staining for NGF immunoreactivity. Inhibitory oligonucleotide delivered by liposomes 3(C) reduced green NGF immunoreactivity in urothelium compared to the control rat treated with saline 3(B) (Magnification 200×).

### DETAILED DESCRIPTION

Underactive bladder (UAB), also referred to as detrusor underactivity, is a chronic, complex and debilitating disease with serious consequences to quality of life. Patients with an UAB have a diminished sense of when the bladder is full and are not able to contract the muscles sufficiently, resulting in incomplete bladder emptying. The current standard pharmacotherapy for UAB is systemic administration of parasympathomimetics, such as oral bethanechol or distigmine, which are often ineffective and have significant systemic side effects due to their pharmacological action on other tissue sites. Therefore, there is an unmet medical need for safe and effective UAB treatment.

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices and materials are now described. All publications mentioned herein are incorporated by reference in their entirety. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to a “lipid” is a reference to one or more lipids and equivalents thereof known to those skilled in the art, and so forth.

It is to be understood that any examples provided in the description are not limiting. For instance, the use of the term “for example” means “for example, but not limited to”, the use of the term “including” means “including, but not limited to”, and the use of the term “include(s)” means “includes, but is not limited to” and so forth throughout the description.

As used herein, the following definitions apply:

“About” means plus or minus 10% of the numerical value of the number with which it is being used. Therefore, about 50% means in the range of 40-60%.

“Administering” means to deliver a therapeutic agent into or onto a target tissue or organ, or to cause a therapeutic agent to be delivered into or onto a target tissue or organ. Thus, as used herein, the term “administering”, when used in conjunction with liposomes, can include providing a liposome into or onto the target tissue and/or organ. Locally administering a composition may be accomplished by topical application, intravesicular instillation, drug eluting matrix, other drug eluting devices or by either method in combination with other known techniques.

The terms “individual”, “host”, “subject”, “patient”, and “animal” may be used interchangeably herein, and include humans and non-human vertebrates such as wild, domestic and farm animals.

“Improves”, as used herein, is used to convey that the present invention changes the appearance, form, characteristics, physiological, and/or physical attributes of the tissue and/or organ to which it is provided, applied, and/or administered.

“Inhibiting”, as used herein, means to prevent the onset of symptoms, alleviate symptoms, decrease symptoms either partially or completely, and/or eliminating the disease, condition and/or disorder.

“Pharmaceutically acceptable” means that the carrier, diluent, and/or excipient in a formulation is compatible with the other ingredients of the formulation and not deleterious to the recipient thereof.

“Therapeutic agent” means an agent utilized to treat, combat, ameliorate, prevent and/or improve an unwanted condition, disease, and/or symptom of a patient.

The “therapeutically effective amount” or “effective amount” of a composition means a predetermined amount calculated to achieve the desired effect, for instance improving the function of the bladder, increasing voiding, improving bladder emptying and/or decreasing urinary retention. The specific dose of a compound administered according to this invention to obtain therapeutic effects will be determined by the particular circumstances surrounding the case, including, for example, the compound administered, the route of administration, and the condition treated. The compounds are effective over a wide range of dosage and, for example, dosages per day will normally fall within the range of from about 0.001 to about 10 mg/kg, more usually in the range of from 0.01 to 1 mg/kg. However, it will be understood that the effective amount administered will be determined by the physician in the light of the relevant circumstances including the condition to be treated, the choice of compound to be administered and the chosen route of administration, and therefore the above dosage ranges are not intended to limit the scope of the invention in any way. A therapeutically effective amount of compound of this invention is typically an amount such that when it is administered in a physiologically tolerable therapeutic composition, it is sufficient to achieve an effective systemic concentration or local concentration in the tissue/organ of interest to improve the disease, condition, and/or disorder.

The terms “treat”, “treated”, or “treating” can be used interchangeably and refer to both therapeutic treatment and preventative measures, wherein the object is to prevent or slow down an undesired physiological condition, disorder, disease, and/or symptom, or to obtain beneficial or desired clinical results. For the purposes of this invention, beneficial or desired clinical results include alleviation of symptoms, diminishment of the extent of the condition, disorder, disease, and/or symptoms, stabilization of the state of the condition, disorder, disease, and/or symptoms, delay in onset or slowing of the progression of the condition, disorder, disease, and/or symptoms, amelioration of the condition, disorder, disease, and/or symptoms, and/or remission (whether partial or total), whether detectable or undetectable, or enhancement or improvement of the condition, disorder, disease, and/or symptoms. Treatment includes eliciting a clinically significant response without excessive levels of side effects. Treatment also includes improving quality of life and/or prolonging survival as compared to expected survival if not receiving treatment.

The terms “carrier”, “excipient”, “diluent”, and “adjuvant” may be used interchangeably and refer to a composition with which the therapeutic agent is administered that is not a therapeutic agent. Such carriers may be sterile liquids such as, for example, water and oils, including those of petroleum, animal, vegetable or synthetic origin. Saline solution, aqueous dextrose and glycerol solution may also be employed as liquid carriers. Suitable pharmaceutical excipients include glucose, starch, lactose, sucrose, gelatin, malt, rice, flour, chalk, sodium chloride, dried skim milk, glycerol, propylene, glycol, water, and ethanol. The composition, if desired, may contain minor amounts of wetting or emulsifying agents, or pH buffering agents. These compositions may take a form of solutions, suspensions, emulsions, powders, sustained-release formulations, or any other pharmaceutically acceptable form.

“Bladder” refers to a urinary bladder which is the organ that collects urine excreted by the kidneys before disposal by urination. A bladder is a hollow, muscular and distensible organ which, in humans, sits on the pelvic floor. Urine enters the bladder via the ureters and exits via the urethra. The bladder wall is comprised of a detrusor muscle, layered around the bladder wall. The detrusor muscle is made of smooth muscle fibers arranged in spiral, longitudinal and circular bundles. The parasympathetic nervous system sends a signal to the detrusor muscle to contract when the bladder wall is stretched. For the urine to exit the bladder, both the internal sphincter and the external sphincter must be opened. The internal sphincter is autonomically controlled, whereas the external sphincter is voluntarily controlled. Complete bladder emptying is dependent on the central nervous system, detrusor muscle activity, coordinated bladder and urethral sphincter function and voluntary initiation of voiding. Dysfunction in any of these processes may result in incomplete bladder emptying.

“Underactive bladder syndrome” (UAB), also known as detrusor underactivity, is a urological condition characterized by bladder underactivity causing difficulty in voiding, resulting in incomplete bladder emptying and urinary retention. As used herein, “UAB” refers to underactive bladder syndrome, detrusor underactivity, and the signs and symptoms associated therewith. In some instances, the symptoms of UAB may include urinary retention, incomplete bladder emptying, slow or intermittent stream, hesitance, terminal dribbling, and straining to urinate. UAB may be caused by bladder outlet obstruction, abnormal bladder contractility, or both. Bladder outlet obstruction may be anatomical, for instance in benign prostatic hyperplasia (BPH) or urethral stricture, or it may be functional, for instance in paruresis or idiopathic detrusor sphincter dyssynergia. Abnormal bladder contractility may result from detrusor muscle underactivity and/or impaired contractility, which may be caused by various factors including sensory failure, myogenic failure, neurogenic defects, neurological diseases affecting the nerves to and from the bladder, side effects of certain drugs, or combinations thereof. Detrusor muscle underactivity and/or impaired contractility caused by sensory failure may be due to diabetes, which can result in sensory and autonomic polyneuropathy. Diabetes may cause damage to neurons innervating the bladder, which may result in bladder dysfunction characterized by impaired sensation of bladder fullness, increased bladder capacity, reduced detrusor contractility, and increased residual urine volume. In some instances, detrusor muscle underactivity is caused by damage to or degeneration of the detrusor muscle. In other instances, detrusor muscle underactivity may be caused by neurogenic factors and/or defects. Detrusor muscle underactivity may be a result of abnormality in the central nervous system when the neurologic pathways innervating the bladder are damaged, for example, in the case of spinal cord injury, in particular injuries to the cauda equina or peripheral sacral nerves. Injuries to cauda equina or peripheral sacral nerves may result from pelvic fractures, transverse sacral fractures, or pelvic plexus injuries. Other injuries that may cause detrusor muscle underactivity include herniated disc(s) or surgical procedures, for example abdominal perineal resection, radical hysterectomy, or proctocolectomy. Neurologic conditions and/or diseases, for example lumbosacral radiculopathy, may cause impaired detrusor muscle contractility. Chronic bladder overdistension caused by a neurologic disease may also result in irreversible detrusor muscle damage even after the treatment of the neurologic disease. Other neurogenic factors and/or diseases that may cause detrusor muscle underactivity include infections of the central nervous system caused by, for example, AIDS, neurosyphilis, herpes zoster, herpes simplex, and Lyme disease. In some cases, side effects from medical therapies, for example anticholinergic medications, may cause blocking of neurotransmission of acetylcholine and result in urinary retention by insufficient activity of the detrusor muscle.

“Pharmaceutically acceptable vehicle” means any conventional pharmaceutical auxiliary substance(s) and/or additives for the formulation of pharmaceutical compositions, for instance pharmaceutically acceptable buffers, preservatives, reducing agents, antioxidants, UV absorbers, stabilizers, penetration promoters, emulsifiers, gelling agents, thickeners, surfactants, colorants, and combinations thereof. Such auxiliary substances and additives are generally preferably pharmaceutically safe. As used herein, “pharmaceutically acceptable vehicle” may include, for example, a lipid vehicle(s).

“Lipid vehicle” means a pharmaceutically acceptable vehicle including fatty or waxy organic compound that is readily soluble in a nonpolar solvent but not in a polar solvent. As used herein, “lipid vehicle” may include, for example, micelles, microemulsions, macroemulsions, liposomes, and the like.

“Micelle” means a colloidal aggregate of amphipathic (surfactant) molecules which are formed at a concentration known as the critical micelle concentration. Micelles are oriented with the non-polar portions at the interior and the polar portions at the exterior surface, exposed to water. The typical number of aggregated molecules in a micelle is about 50 to about 100.

“Microemulsion” means a thermodynamically stable, isotropic liquid mixture of lipid, water, and surfactant, frequently in combination with a cosurfactant. The aqueous phase may contain salt(s) and/or other ingredients, and the lipid may be a mixture of different hydrocarbons and olefins. A microemulsion may form upon simple mixing of the components (spontaneous formation) and may not require the high shear conditions generally used in the formation of ordinary emulsions. As used herein, a “microemulsion” is essentially a swollen micelle, however not all solutions containing micelles can be swollen to form micoremulsions. A microemulsion typically has a droplet diameter of about 10 about 100 nm.

“Macroemulsion” means a kinetically stable mixture of at least two immiscible liquids, wherein at least one of the liquids has a droplet diameter of greater than about 100 nm.

“Liposome” means a spherical vesicle, which may be artificially prepared, composed of a lipid bilayer which can be used as a vehicle for the administration (delivering) of a therapeutic agent. A liposome may be a closed lipid vesicle composed of concentric lipid bilayers enclosing an aqueous interior. The lipid vesicle may comprise one or more than one aqueous compartment delineated by one (unilamellar) or more than one (multi-lamellar) lipid bilayer(s). Lipids comprising a liposome may include lipids used as therapeutic agents. A liposome may include cationic lipids and/or non-ionic lipids. A therapeutic agent may be enclosed inside a liposome, partially enclosed inside a liposome, and/or attached to the outside of a liposome. Liposomes may be produced by any known method, including dehydration methods, dehydration-rehydration methods, and/or micro fluidization.

Dehydrated liposomes may be formed from a homogenous dispersion of phospholipid in a in a tert-butyl alcohol (TBA)/water co-solvent system. The isotropic monophasic solution of liposomes is freeze dried to generate dehydrated liposomal powder in a sterile vial. The freeze drying step leaves empty lipid vesicles or dehydrated liposomes after removing both water and TBA from the vial. On addition of physiological saline, the lyophilized product spontaneously forms a homogenous liposome preparation. Low lipid concentrations work ideally for this method because lipid and TBA ratio is the key factor affecting the size and the polydispersity of resulting liposome preparation.

Liposomal oligonucleotides may be prepared by modified dehydration-rehydration method. Prepared liposomes may be hydrated with a solution of oligonucleotides in water for injection (50 units/ml) at 25° C. The mixture may then be incubated for 30-120 minutes at 25° C. using a water bath to form oligolamellar hydration liposomes. Mannitol may be added to the final mixture at a concentration of 0.5%, 1%, 2.5%, or 5% mannitol (w/v), before freezing in acetone-dry ice bath. Mannitol acts as a cryoprotectant in the freeze drying process. The frozen mixture may then be lyophilized at −40° C. and 5 millibar overnight. The lyophilized cake may be resuspended with saline to the desired final concentration of oligonucleotides. The free oligonucleotides may be removed from entrapped oligonucleotides by centrifugation at 12,000×g for 30 minutes using an ultracentrifuge. After washing three times, the precipitates may again be resuspended in saline.

Liposomes may be used as a vehicle for delivering therapeutic agents including acetylcholinesterase inhibitors, cannabinoids, muscarinic receptor agonists, muscarinic receptor modulators, neuroprotective agents, ghrelin receptor agonists, neurotrophins, potassium channel blockers, prostanoid agonists, and oligonucleotides.

“Lipid” means a fatty or waxy organic compound that is readily soluble in a nonpolar solvent but not in a polar solvent. As used herein, “lipid” may refer to any specific compound belonging to one or more classes of lipids including phospholipids, glycolipids, sphingolipids, sphingophospholipids, or sphingoglycolipids. It is to be understood that the classification of lipids is not mutually exclusive. For example, in some instances, a glycolipid may also be a phospholipid and in other instances, a phospholipid may also be a glycolipid.

“Phospholipid” means a lipid molecule having a hydrophilic “head” containing a negatively charged phosphate group and a hydrophobic “tail” usually comprising a long fatty acid hydrocarbon chain. The hydrophilic head may further comprise polar groups other than phosphate. As used herein, “phospholipid” may include any natural or synthetic phospholipid, for example phosphatidylcholine, phosphatidic acid, phosphatidylethanolamine, phosphatidylserine, phosphatidylinositide, phosphatidylglycerol, phosphosphingolipids, cardiolipin, sphingomyelin, sphingosine I-phosphate, ceramidegalactopyranoside, ganglioside, cerebroside, cholesterol, 1,2-distearoylsn-glycero-3-phosphocholine, or 1,2-dioleoylphosphatidylcholine.

“Glycolipid” means an oligosaccharide (carbohydrate) covalently attached to a lipid. As used herein, “glycolipid” may include any natural or synthetic glycolipid, for example galactolipids, sulfolipids, glycosphingolipids, cerebrosides, galactocerebrosides, glucocerebrosides, sulfatides, gangliosides, globosides, glycophosphosphingolipids, or glycosylphosphatidylinositols.

“Sphingolipid” means a lipid containing a backboane of sphingoid bases, which are a set of aliphatic amino alcohols including sphingosine.

“Sphingophospholipid” means a lipid containing one fatty acid and one phosphate group attached to a sphigosine molecule and an alcohol attached to the phosphate group

“Sphingoglycolipid” means a lipid containing at least one monosaccharide residue and either a sphingoid or a ceramide.

“Acetylcholinesterase inhibitor” means a natural or synthetic compound that exhibits inhibition of acetylcholinesterase. Inhibition may be reversible, irreversible, or quasi-irreversible.

“Cannabinoid” means any of various chemical constituents of cannabis.

“Muscarinic receptor agonist” and “muscarinic receptor modulator” refer to any agent that modulates or stimulates the activity of the acetylcholine muscarinic receptor.

“Prostanoid receptor agonist” means any member of the eicosanoid family of phospholipid mediators that binds to the prostanoid receptor and activates the cyclooxygenase pathway. Prostanoid-receptor agonists can possess antagonistic activity, in addition to agonistic activity, even within the same target tissue. As used herein, “prostanoid receptor agonist” may refer to any chemical compound that is a structural derivative or analogue of a prostaglandin or thromboxane. Such prostanoid-receptor agonists include prostaglandin E1 (PGE1) derivatives such as limaprost, gemeprost, and misoprostol. The term “prostaglandin” includes natural and synthetic analogs and derivatives of prostaglandins which exhibit functional and physical responses similar to those of prostaglandins per se.

“Oligonucleotide” means a short, single stranded DNA or RNA molecule. “Inhibitory oligonucleotide” means an oligonucleotide specific for inhibiting a target DNA or RNA sequence. Representative inhibitory oligonucleotides include dsRNA, siRNA, shRNA, miRNA, piRNA, external guide sequences, ribozymes, and other short catalytic RNAs. Expression of the target nucleic acid can be inhibited at the transcriptional level or translational level.

Some aspects of the present invention are directed to methods of treating an underactive bladder and/or symptoms thereof by locally administering a composition comprising a pharmaceutically acceptable vehicle and an effective amount of a therapeutic agent. Some aspects of the present invention are directed to compositions for treating an underactive bladder and/or symptoms thereof including a pharmaceutically acceptable vehicle and an effective amount of a therapeutic agent. The following embodiments apply to any of the compositions and/or methods described herein.

In some embodiments, the method for treating an underactive bladder condition and/or symptoms thereof includes locally administering to the bladder a composition including a pharmaceutically acceptable lipid vehicle and an effective amount of a therapeutic agent, wherein the underactive bladder condition and/or symptoms thereof are treated.

In some embodiments, the underactive bladder condition and/or symptoms thereof is selected from urinary retention, incomplete bladder emptying, slow or intermittent stream, hesitancy, terminal dribbling, straining to urinate that is related to diabetes, neurological diseases affecting the nerves to and from the bladder, bladder outlet obstruction and impaired contractility of detrusor muscle and combinations thereof.

In some embodiments, the underactive bladder condition or symptoms thereof is caused by a condition selected from benign prostate hyperplasia, urethral stricture, paruresis, detrusor muscle underactivity, underactive bladder syndrome, anticholinergic drug effect, bladder sensory dysfunction, diabetes and combinations thereof.

In some embodiments, the effective amount of the therapeutic agent is an amount less than the amount administered systemically to obtain the same therapeutic efficacy.

In some embodiments, the local administration is selected from instillation, drug eluting matrix, drug eluting device, topical and combinations thereof.

In some embodiments, the composition further includes a physiologically acceptable carrier, excipient or diluent.

In some embodiments, the lipid vehicle includes one or more of the following: a phospholipid, a glycolipid, a sphingolipid, a sphingophospholipid, a sphingoglycolipid, and combinations thereof.

In some embodiments, the lipid vehicle comprises one or more of the following: phosphatidylcholine, phosphatidylethanolamine, phosphatidylserine, phosphatidylinositol, phosphatidylglycerol, cardiolipin, sphingomyelin, sphingosine I-phosphate, ceramidegalactopyranoside, ganglioside, cerebroside, cholesterol, 1,2-distearoylsn-glycero-3-phosphocholine, 1,2-dioleoylphosphatidylcholine and combinations thereof.

In some embodiments, the therapeutic agent is selected from an acetylcholinesterase inhibitor, a potassium channel blocker, a cannabinoid, a muscarinic receptor agonist, a muscarinic receptor modulator, a neurotrophin, a ghrelin receptor agonist, a neuroprotective agent, a prostanoid receptor agonist, an inhibitory oligonucleotide molecule, and combinations thereof.

In some embodiments, the therapeutic agent includes an acetylcholinesterase inhibitor. In some embodiments, the acetylcholinesterase inhibitor is selected from carbamates, organophosphates, phenantherene derivatives, piperidines, phyostigmine, neostigmine, rivastigmine, pyridostigmine, ambenonium, demarcarium, tacrine, donepezil, distgmine, phenserine, galantamine, edrophonium, huperzine A, ladostigil, ungeremine, lactucopicrin, and their pharmaceutically accepted salts and solvates.

In some embodiments, the acetylcholinesterase inhibitor includes a carbamate. In some embodiments, the carbamate includes aldicarb, bendiocarb, bufencarb, carbaryl, carbendazim, carbetamide, carbofuran, carbosulfan, chlorbufam, chloropropham, ethiofencarb, formetanate, methiocarb, methomyl, oxamyl, phenmedipham, pinmicarb, pirimicarb, propamocarb, propham, and propoxur, and their pharmaceutically accepted salts and solvates, or combinations thereof.

In some embodiments, the acetylcholinesterase inhibitor includes an organophosphate. In some embodiments, the organophosphate includes echothiophate, diisopropyl fluorophosphate, cadusafos, cyclosarin, dichlorvos, dimethoate, metrifonate, parathion, malathion, diazinon or their pharmaceutically accepted salt or solvate, or combinations thereof.

In some embodiments, the acetylcholinesterase inhibitor is selected from diisopropyl fluorophosphates, phospholine iodide (echothiophate), parathion, malathion, diazinon, pharmaceutically acceptable salts thereof, pharmaceutically acceptable solvates thereof, and combinations thereof.

In some embodiments, the acetylcholinesterase inhibitor is selected from physostigmine, neostigmine, rivastigmine, pyridostigmine, ambenonium, demarcarium, tacrine, donepezil, distigmine, phenserine, galantamine, edrophonium, huperzine A, ladostigil, ungeremine, lactucopicrin, pharmaceutically acceptable salts thereof, pharmaceutically acceptable solvates thereof, and combinations thereof.

In some embodiments, the therapeutic agent includes a potassium channel blocker.

In some embodiments, the therapeutic agent includes a cannabinoid. In some embodiments, the cannabinoid includes Δ⁹-tetrahydrocannabinol, a synthetic cannabinoid, a semi synthetic cannabinoid, or combinations thereof.

In some embodiments, the therapeutic agent includes a muscarinic receptor agonist and/or a muscarinic receptor modulator. In some embodiments, the muscarinic receptor agonist and/or muscarinic receptor modulator includes sabcomeline, milameline, xanomeline, oxotremorine, pilocarpine, or pharmaceutically acceptable salts or solvates thereof, or combinations thereof.

In some embodiments, the therapeutic agent includes a neurotrophin. In some embodiments, the neurotrophin includes nerve growth factor (NGF), brain-derived neurotrophic factor (BDNF), neurotrophin-3 (NT-3), neurotrophin-4 (NT-4), glial cell-line derived neurotrophic factor (GDNF), ciliary neurotrophic factor (CNTF), analogs thereof, homologs thereof, or combinations thereof.

In some embodiments, the therapeutic agent includes a ghrelin receptor agonist. In some embodiments, the ghrelin receptor agonist includes capromorelin, hexarelin, ipamorelin, tabimorelin, or pharmaceutically acceptable salts or solvates thereof, or combinations thereof.

In some embodiments, the therapeutic agent includes a neuroprotective agent. In some embodiments, the neuroprotective agent includes acetyl-L-carnitine, xaliproden, cerebrolysin, or combinations thereof.

In some embodiments, the therapeutic agent includes a prostanoid receptor agonist. Examples of suitable prostanoid-receptor agonists include, but are not limited to, prostaglandins, such as those of the prostaglandin E class (PGE) and prostaglandin F class (PGF), and thromboxanes. Suitable members of the PGE class include, but are not limited to, PGE1 and PGE2. Additionally, suitable members of the PGF class include, but are not limited to, PGF_{1α} and PGF₂ₐ. In some embodiments, the prostanoid receptor agonist includes prostaglandin E1, limaprost, gemeprost, misotprostol, prostaglandin E2, dinoprostone, sulprostone, prostaglandin F₂ₐ, carboprost, or combinations thereof.

In some embodiments, the therapeutic agent includes an inhibitory oligonucleotide. In some embodiments, the inhibitory oligonucleotide may have a base sequence substantially complementary to a sequence of a target mRNA or a target mRNA precursor. In some embodiments, the target RNA encodes acetylcholinesterase or an acetylcholinesterase protein. In some embodiments, the target RNA encodes an acetylcholinesterase activator. In some embodiments, the target RNA inhibits formation of acetylcholine. In some embodiments, the target RNA attenuates muscarinic receptors. In some embodiments, the target RNA attenuates cannabinoid receptors. In some embodiments, the target RNA attenuates ghrelin receptors. In some embodiments, the target RNA attenuates prostanoid receptors. In some embodiments, the target RNA encodes potassium channel receptors, or encodes for a potassium channel agonist. In some embodiments, the inhibitory oligonucleotide molecule is selected from a dsRNA, siRNA, shRNA, miRNA, piRNA, external guide sequences, ribozymes, short catalytic RNAs, and combinations thereof.

Inhibitory RNAs may contain any known base analogs including, but not limited to, 4-acetylcytosine, 8-hydroxy-N-6-methyladenosine, aziridinylcytosine, pseudoisocytosine, 5-(carboxyhydroxylmethyl) uracil, 5-bromouracil, 5-carboxymethylaminomethyl-2-thiouracil, 5-carboxymethylaminomethyluracil, dihydrouracil, inosine, N6-isopentenyladenine, 1-methyladethne, 1-methylpseudouracil, 1-methyl guanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N 6-isopentenyladenine, uracil-5-oxyacetic acid methyl ester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methyl ester, 2,6-diaminopurine, and 2′-modified analogs such as, but not limited to 0-methyl, amino-, and fluoro-modified analogs.

Inhibitory RNAs are configured to hybridize to target mRNAs or mRNA precursors and modulate their expression through several means, including directly catalyzing target mRNA degradation, causing the recruitment of cellular proteins and enzymes that mediate mRNA degradation, inhibiting or reducing the translation of target mRNA, or otherwise reducing the stability of target mRNA. Inhibitory RNAs can be single stranded or double stranded. Exemplary inhibitory RNAs include, but are not limited to, dsRNA, siRNA, shRNA, miRNA, piRNA, external guide sequences, ribozymes, and other short catalytic RNAs.

Inhibitory RNAs are complementary to their target RNAs. The term “complementary”, as used herein, refers to the capacity of two nucleotides to pair precisely with each other. This term may also be used to refer to oligonucleotides which exhibit the ability of pairing precisely with each other. For example, if the nucleotides located at a certain position on two oligonucleotides are capable of hydrogen bonding, then the oligonucleotides are considered to be complementary to each other at that position. The inhibitory RNAs and the target RNAs are complementary to each other when a sufficient number of corresponding positions in each molecule are occupied by nucleotides which can hydrogen bond with each other. Thus, “complementary” is a term that is used to indicate a sufficient degree of complementarity or precise pairing such that stable and specific binding may occur between the inhibitory RNA and the target RNA. It is understood in the art that the sequence of an inhibitory oligonucleotide compound need not be 100% complementary to that of its target RNA. A sufficient degree of complementarity prevents non-specific binding of the inhibitory oligonucleotide compound to non-target sequences under conditions in which specific binding is desired, i.e. under physiological conditions.

Oligodeoxynucleotides can include or be a phosphodiester backbone, a phosphothioate backbone, locked nucleic acid, peptide nucleic acid, tricycle-DNA, decoy oligonucleotide, ribozymes, spiegelmers (containing L nucleic acids, an aptamer with high binding affinity), or CpG oligomers.

In one embodiment, the inhibitory RNAs may be siRNAs or shRNAs. The term “siRNA” means a small interfering RNA that is a short-length double-stranded RNA that is not toxic. Generally, there is no particular limitation in the length of siRNA as long as it does not show toxicity. “siRNAs” can be, for example, 15 to 49 bp. In some instances, the siRNAs may be 15 to 35 bp, and in other instances the siRNAs 21 to 30 bp long. Inhibitory nucleic acids and methods of producing them are well known in the art. siRNA design software is available, for example, at http://i.cs.hku.hk/-sirna/software/sirna.php. The sequence of at least one strand of the siRNA contains a region complementary to at least a part of the target mRNA sufficient for the siRNA to specifically hybridize to the target mRNA. In one embodiment, the siRNA may be substantially identical to at least a portion of the target mRNA. “Identity”, as known in the art, is the relationship between two or more polynucleotide (or polypeptide) sequences, as determined by comparing the sequences. In the art, identity also means the degree of sequence relatedness between polynucleotide sequences, as determined by the match between strings of such sequences. Despite there being numerous methods for measuring “identity”, the term “Identity” is well known to skilled artisans. Methods are commonly employed to determine identity between sequences. Preferred methods to determine identity are designed to give the largest match between the sequences tested. Methods to determine identity are codified in computer programs such as, for example, GCG program package, BLASTP, BLASTN, PASTA, and CLUSTAL. The CLUSTAL program compares the sequences of two polynucleotides and finds the optimal alignment by inserting spaces in either sequence as appropriate. A software package such as BLASTx can be used to calculate the identity for an optimal alignment. One skilled in the art will appreciate that two polynucleotides of different lengths may be compared over the entire length of the longer fragment. Alternatively small regions may be compared. Normally sequences of the same length are compared for a useful comparison to be made.

In one embodiment, the inhibitory nucleic acid has 100% sequence identity with at least a part of the target mRNA. However, inhibitory nucleic acids having 70%, 80% or greater than 90% or 95% sequence identity may be used. Thus, sequence variations that might be expected due to genetic mutation, strain polymorphism, or evolutionary divergence can be tolerated.

The duplex region of the RNA may have a nucleotide sequence that is capable of hybridizing with a portion of the target gene transcript.

The double-stranded RNA portions of siRNAs in which two RNA strands pair up are not limited to the completely paired ones, and may contain nonpairing portions due to mismatch (the corresponding nucleotides are not complementary) or bulge (lacking in the corresponding complementary nucleotide on one strand). Nonpairing portions can be contained to the extent that they do not interfere with siRNA formation. Suitable siRNAs can contain one or more modified bases, or have a modified backbone to increase stability or for other reasons. For example, the phosphodiester linkages of natural RNA may be modified to include at least one of a nitrogen or sulfur heteroatom. siRNAs comprising unusual bases, including, but not limited to inosine, or modified bases, such as tritylated bases, can be used. The term “siRNA”, as used herein, includes such chemically, enzymatically or metabolically modified forms of siRNA.

The terminal structure of siRNA may be either blunt or cohesive (overhanging) as long as siRNA can silence, reduce, or inhibit the target gene expression due to its RNAi effect. The cohesive (overhanging) end structure is not limited only to the 3′ overhang, and the 5′ overhanging structure may be included as long as it is capable of inducing the RNAi effect. The number of overhanging nucleotide can be any numbers as long as the overhang is capable of inducing the RNAi effect. For example, the overhang can consist of 1 to 8, or in some instances 2 to 4 nucleotides.

The terminal structure of the siRNA is not necessarily the cut off structure at both ends as described above, and may have a stem-loop structure in which ends of one side of double-stranded RNA are connected by a linker RNA. siRNAs containing a linker RNA that forms a hairpin structure are referred to as short hairpin RNAs, or shRNAs. The length of the double-stranded RNA region (stem-loop portion) can be, for example, 15 to 49 bp, or 15 to 35 bp, or 21 to 30 bp long. Alternatively, the length of the double-stranded RNA region that is a final transcription product of siRNAs to be expressed is, for example, 15 to 49 bp, or 15 to 35 bp, or 21 to 30 bp long. Furthermore, there is no particular limitation in the length of the linker as long as it has a length so as not to hinder the pairing of the stem portion. For example, for stable pairing of the stem portion and suppression of the recombination between DNAs coding for the portion, the linker portion may have a clover-leaf tRNA structure. Even though the linker has a length that hinders pairing of the stem portion, it is possible, for example, to construct the linker portion to include introns so that the introns are excised during processing of precursor RNA into mature RNA, thereby allowing pairing of the stem portion.

Micro RNAs (referred to as “miRNAs”) are small non-coding RNAs, belonging to a class of regulatory molecules found in plants and animals that control gene expression by binding to complementary sites on target messenger RNA (mRNA) transcripts. miRNAs are generated from large RNA precursors (termed pre-miRNAs) that are processed in the nucleus into approximately 70 nucleotide pre-miRNAs, which fold into imperfect stem-loop structures. The pre-miRNAs undergo an additional processing step within the cytoplasm where mature miRNAs of 18-25 nucleotides in length are excised from one side of the pre-miRNA hairpin by an RNase III enzyme, Dicer. MiRNAs have been shown to regulate gene expression in two ways. First, miRNAs that bind to protein-coding mRNA sequences that are exactly complementary to the miRNA induce the RNA mediated interference (RNAi) pathway. Messenger RNA targets are cleaved by ribonucleases in the ribonucleoprotein complex known as the RISC complex. This mechanism of miRNA-mediated gene silencing has been observed in plants and in animals. In the second mechanism, miRNAs that bind to imperfect complementary sites on messenger RNA transcripts direct gene regulation at the post-transcriptional level but do not cleave their mRNA targets. MiRNAs identified in both plants and animals use this mechanism to exert translational control of their gene targets.

In some embodiments, miRNAs have at least 80%, at least 90%, or at least 95% sequence identity with the target mRNA. Naturally occurring microRNAs that regulate target RNAs, pri-miRNA, pre-miRNA, mature miRNA or fragments of variants thereof that retain the biological activity of the mature miRNA and DNA encoding a pre-miRNA, pre-miRNA, mature miRNA, fragments or variants thereof, or regulatory elements of the miRNA, have been identified. The size of the miRNA may range from 21 nucleotides to 170 nucleotides, although nucleotides of up to 2000 nucleotides can be utilized. In some embodiments, the size range of the pre-miRNA may be from 70 to 170 nucleotides in length and the mature miRNA may be from 21 to 25 nucleotides in length.

A class of 24- to 30-nt RNAs may be generated by a Dicer-independent mechanism wherein the RNAs interact with a subset of Argonaute proteins related to Piwi. Studies in Drosophila have identified five Argonaute proteins: Ago1, Ago2, Ago3, Piwi, and Aubergine (Aub). Ago1 and Ago2 are ubiquitously expressed, whereas the expression of Piwi, Aub, and Ago3 are germ line-specific. Ago1 associates with miRNAs to regulate endogenous gene expression, and Ago2 serves as the slicer for siRNA-mediated gene silencing. Piwi, Aub, and Ago3 have been recently reported to interact with 24- to 30-nt small RNAs known as rasiRNAs. Murine Ago1, Ago2, Ago3, and Ago4 are associated with miRNAs, while the Piwi orthologs, MIWI, MILI, and MIWI2, are found in germ line cells.

Like other members of the Ago family, Piwi proteins associate with small RNAs that act as guides in silencing target mRNA. These Piwi-interacting RNAs are called piRNAs. These small RNAs associated with Piwi RNPs have been cloned and sequence analysis of piRNAs shows a high percentage of uridine residues at the 5′ termini, and genomic mapping shows that piRNAs are concentrated at a few loci.

Primary transcripts for piRNAs are generated from the transposon regulatory regions of heterochromatin. These piRNAs are anti-sense, or complementary to transposon transcripts, and associated with both Piwi and Aub to trigger the amplification loop. Piwi/Aub cleaves target transposon transcripts between 10 and 11 nt from the 5′ end of anti-sense piRNA and subsequently generates Ago3-associated sense piRNA. Ago3 functions as another slicer, which recognizes the complementary sequence of piRNA cluster transcripts, and generates more Piwi/Aub-associated anti-sense strand piRNA. piRNAs are thought to function with Piwis endogenously to maintain transposon silencing.

Ribonuclease P (RNase P) is a ribonucleoprotein complex found in all organisms. It is highly active in cells and is responsible for the maturation of 5′ termini of all tRNAs, which account for approximately 2% of total cellular RNA. Human RNase P has at least nine polypeptides and a RNA subunit (HI RNA). One of the unique features of RNase P is its ability to recognize structures, rather than the sequences, of substrates. This allows RNase P to hydrolyze different natural substrates in vivo or in vitro. Accordingly, any complex of two RNA molecules that resembles a tRNA molecule can be recognized and cleaved by RNase P. One of the RNA molecules is called the external guide sequence (EGS). An mRNA sequence can be targeted for RNase P cleavage by using EGSs to hybridize with the target RNA and direct RNase P to the site of cleavage. The EGSs used to direct human RNase P for targeted cleavage resemble three-quarters of a tRNA molecule and consist of two sequence elements: a targeting sequence complementary to the mRNA sequence and a guide sequence, which is a portion of the natural tRNA sequence and is required for RNase P recognition.

An EGS is designed to base pair through hydrogen bonding mechanism with a target mRNA to form a molecular structure similar to that of a transfer RNA (tRNA). The EGS/mRNA target is then cleaved and inactivated by RNAse P. EGS are not consumed in this reaction, but instead can recycle as a catalyst through multiple cycles of target mRNA cleavage leading to target inactivation more effectively than conventional anti-sense DNA oligonucleotides. EGS combine the specificity of conventional antisense DNA for gene targeting with the catalytic potency of RNAse P. RNase P is present in abundant quantities in all viable eukaryotic cells where it serves to process transfer RNA (tRNA) by cleavage of a precursor transcript.

Small RNA sequences have been described that target eukaryotic mRNA for degradation by endogenous RNase P, a ubiquitous cellular enzyme that generates mature transfer RNA (tRNA) from precursor transcripts. A small RNA termed an External Guide Sequence (EGS) can be designed that mimics certain structural features of a tRNA molecule when it forms a bimolecular complex with another RNA sequence contained within a cellular messenger RNA (mRNA). Thus, any mRNA can in principle be recognized as a substrate for RNase P with both the EGS and RNase P participating as co-catalysts although due to the complexity of the binding and cleavage steps the kinetics of the reaction are difficult to predict in vitro or in vivo.

Design of an EGS requires both knowledge of the mRNA primary sequence to be cleaved by RNase Pas well as the secondary structure of the mRNA sequences in the mRNA. EGS sequences must be complementary to the primary sequence of the targeted mRNA and the sequences in the mRNA must be exposed in a single-stranded conformation within the mRNA secondary structure in order to bind to the EGS. Secondary structure of target mRNA can be approximated by computer modeling or determined empirically using nucleases or other RNA cleaving reagents well known to one of ordinary skill in the art. This analysis may be useful in locating regions of mRNA for targeting with complementary oligonucleotides including conventional DNA antisense oligonucleotides and catalytic RNA.

RNase P is a ribonucleoprotein having two components, an RNA component and a protein component. The RNA component of RNase P is responsible for the catalytic cleavage which forms the mature 5′ ends of all transfer RNAs. RNase P is endogenous to all living cells that have been examined. During the studies on recognition of substrate by RNase P, it was found that E. coli RNase P can cleave synthetic tRNA-related substrates that lack certain domains, specifically, the D, TψC and anticodon stems and loops, of the normal tRNA structure. For bacterial RNAse P a half-tum of an RNA helix and a 3′ proximal CCA sequence contain sufficient recognition elements to allow the reaction to proceed. Using these principles, any RNA sequence can be converted into a substrate for bacterial RNase P by using an external guide sequence, having at its 5′ terminus nucleotides complementary to the nucleotides 3′ to the cleavage site in the RNA to be cleaved and at its 5′ terminus the nucleotides NCCA (SEQ ID NO: 1) (N is any nucleotide).

EGS for promoting RNase P-mediated cleavage of RNA has also been developed for use in eukaryotic systems. As used herein, “external guide sequence” and “EGS” refer to any oligonucleotide or oligonucleotide analog that forms, in combination with a target RNA, a substrate for RNaseP.

An external guide sequence for promoting cleavage by RNase P contains sequences which are complementary to the target RNA and which forms secondary and tertiary structures similar to portions of a tRNA molecule. In eukaryotes, including mammals, tRNAs are encoded by families of genes that are 73 to 150 base pairs long. tRNAs assume a secondary structure with four base paired stems known as the cloverleaf structure. The tRNA contains a stem, a D loop, a Variable loop, a TψC loop, and an anticodon loop. In one form, the EGS contains at least seven nucleotides which base pair with the target sequence 3′ to the intended cleavage site to form a structure like the stem, nucleotides which base pair to form stem and loop structures similar to the TψC loop, the Variable loop and the anticodon loop, followed by at least three nucleotides that base pair with the target sequence to form a structure like the D loop.

Preferred guide sequences for eukaryotic RNase P consist of a sequence which, when in a complex with the target RNA molecule, forms a secondary structure resembling that of a tRNA cloverleaf or parts thereof. The desired secondary structure is determined using conventional Watson-Crick base pairing schemes to form a structure resembling a tRNA. Since RNAse P recognizes structures as opposed to sequences, the specific sequence of the hydrogen bonded regions is less critical than the desired structure to be formed. The EGS and the target RNA substrate should resemble a sufficient portion of the tRNA secondary and tertiary structure to result in cleavage of the target RNA by RNase P. The sequence of the EGS can be derived from any tRNA. The consensus sequence for RNase P recognition of tRNA molecules is GNNNNNU (SEQ ID NO:2). The sequence obtained from the stem of the tRNA is altered to be complementary to the identified target RNA sequence. Target RNA is mapped by techniques well known to one of ordinary skill in the art for the consensus sequence. Such techniques include digestion of the target mRNA with T1 nuclease. Digestion with T1 nuclease cleaves RNA after guanine (G) residues that are exposed in solution and single-stranded, but not after G residues that are buried in the RNA secondary structure or base paired into double stranded regions. The reaction products form a ladder after size fractionation by gel-electrophoresis. A T1 sensitive site is detected as a dark band is compared to the target mRNA sequence to identify RNase P consensus sequences. The complimentary sequence from the target mRNA is used for the EGS. The complementary sequences may consist of as few as seven nucleotides, but preferably include eleven nucleotides, in two sections which base pair with the target sequence and which are preferably separated by two unpaired nucleotides in the target sequence, preferably UU, wherein the two sections are complementary to a sequence 3′ to the site targeted for cleavage.

The remaining portion of the guide sequence, which is required to cause RNase P catalytic RNA to interact with the EGS/target RNA complex, is herein referred to as an RNase P binding sequence. The anticodon loop and the Variable loop can be deleted and the sequence of the TAX loop can be changed without decreasing the usefulness of the guide sequence. External guide sequences can also be derived using in vitro evolution techniques.

In another embodiment, the inhibitory RNA is a catalytic RNA, or a ribozyme. While ribozymes that cleave mRNA at site specific recognition sequences can be used to degrade target mRNAs the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art. There are usually numerous potential hammerhead ribozyme cleavage sites within each nucleotide sequence of a target mRNA of known sequence. Preferably, the ribozyme is engineered so that the cleavage recognition site is located near the 5′ end of the target. mRNA. This functions to increase efficiency and minimize the intracellular accumulation of non-functional mRNA transcripts.

Inhibitory RNAs can be produced using methods known to those skilled in the art. They can be chemically synthesized, produced by in vitro transcription; expressed in cells from an expression plasmid or viral vector; or expressed in cells from a polymerase chain reaction (PCR)-derived expression cassette. In vitro synthesis may be chemical or enzymatic, for example using cloned RNA polymerase (e.g., T3, T7, SP6) for transcription of the endogenous DNA (or eDNA) template, or a mixture of both. SiRNAs can also be produced by digestion of long dsRNA by an RNase III family enzyme (e.g., Dicer, RNase III). In a preferred embodiment, the inhibitory RNAs are obtained synthetically, for example, by chemically synthesizing a nucleic acid by any method of synthesis known to the skilled artisan. The synthesized inhibitory RNAs can then be purified by any method known in the art. Methods for chemical synthesis of nucleic acids include in vitro chemical synthesis using phosphotriester, phosphate or phosphoramidite chemistry and solid phase techniques, or via deoxynucleoside H-phosphonate intermediates.

In vivo, inhibitory RNAs may be synthesized using recombinant techniques well known in the art. For example, bacterial cells can be transformed with an expression vector which comprises the DNA template from which the inhibitory RNAs are to be derived. The RNA can be purified by extraction with a solvent (such as phenol/chloroform) or resin, precipitation (for example in ethanol), electrophoresis, chromatography, or a combination thereof.

Methods for producing inhibitory RNAs that target mRNAs of known sequence are known in the art. One of skill in the art could readily produce inhibitory RNAs that downregulate the expression of any chosen mRNA in host using information that is publicly available. Inhibitory nucleic acids and methods of producing them are well known in the art. siRNA design software is available, for example, at http://i.cs.hkn.hk/-sima/software/sima.php. Synthesis of nucleic acids is well known.

In some embodiments, the inhibitory oligonucleotide molecule includes an antisense oligodeoxynucleotide less than 30 nucleotides in length.

In some embodiments, the inhibitory oligonucleotide molecule has a base sequence substantially complementary to a sequence of a target mRNA.

In some embodiments, the inhibitory oligonucleotide molecule has a base sequence substantially complementary to a sequence of a target mRNA precursor.

In some embodiments, the target mRNA is mRNA complementary to a gene encoding an acetylcholinesterase protein.

In some embodiments, the inhibitory oligonucleotide molecule includes one or more of the following: a phosphodiester backbone, a phosphorothioate backbone, a locked nucleic acid, peptide nucleic acid, tricycle-DNA, a decoy oligonucleotide, a ribozyme, a spiegelmer, a CpG oligomer, and combinations thereof.

In some embodiments, the composition includes wherein the therapeutic agent is an inhibitory oligonucleotide delivered in an acceptable lipid vehicle. In some embodiments, the molar ratio of the lipid comprising the liposome to the inhibitory oligonucleotide may range from about 1:1 to about 20:1. In some embodiments, the inhibitory oligonucleotide may be replaced by another therapeutic agent and conditions modified suitably to form liposomes encapsulating the therapeutic agent. Skilled artisan will readily know the conditions that need to be optimized, depending on the specific molecular structure of the therapeutic agent chosen.

In some embodiments, the lipid vehicle includes a liposome of cationic and non-ionic lipids.

In some embodiments, the therapeutic agent includes an inhibitory oligonucleotide and the lipid vehicle to inhibitory oligonucleotide molecule molar ratio ranges from at least about 1:1 to about 20:1.

In some embodiments, the composition is administered via intravesicular instillation.

In some embodiments, a composition for treating an underactive bladder condition or symptoms thereof includes a pharmaceutically acceptable lipid vehicle and an effective amount of a therapeutic agent for treating the underactive bladder condition or symptoms. In some embodiments, the underactive bladder condition or symptoms thereof is selected from urinary retention, incomplete bladder emptying, slow or intermittent stream, hesitancy, terminal dribbling, straining to urinate that is related to diabetes, neurological diseases affecting the nerves to and from the bladder, bladder outlet obstruction and impaired contractility of detrusor muscle and combinations thereof. In some embodiments, the underactive bladder condition or symptoms thereof is caused by one or more of the following conditions: benign prostate hyperplasia, urethral stricture, paruresis, detrusor muscle underactivity, underactive bladder syndrome, anticholinergic drug effect, bladder sensory dysfunction and diabetes. In some embodiments, the composition further comprises a physiologically acceptable carrier, excipient or diluent. In some embodiments, the lipid vehicle comprises one or more of the following: a phospholipid, a glycolipid, a sphingolipid, a sphingophospholipid, a sphingoglycolipid, and combinations thereof. In some embodiments, the lipid vehicle comprises one or more of the following: phosphatidylcholine, phosphatidylethanolamine, phosphatidylserine, phosphatidylinositol, phosphatidylglycerol, cardiolipin, sphingomyelin, sphingosine I-phosphate, ceramidegalactopyranoside, ganglioside, cerebroside, cholesterol, 1,2-distearoylsn-glycero-3-phosphocholine, 1,2-dioleoylphosphatidylcholine and combinations thereof.

In some embodiments, the composition includes a therapeutic agent selected from an acetylcholinesterase inhibitor, a potassium channel blocker, a cannabinoid, a muscarinic receptor agonist, a muscarinic receptor modulator, a neurotrophin, a ghrelin receptor agonist, a neuroprotective agent, a prostanoid agonist, an inhibitory oligonucleotide molecule, and combinations thereof. In some embodiments, the acetylcholinesterase inhibitor is a carbamate. In some embodiments, the acetylcholinesterase inhibitor is selected from physostigmine, neostigmine, rivastigmine, pyridostigmine, ambenonium, demarcarium, tacrine, donepezil, distigmine, phenserine, galantamine, edrophonium, huperzine A, ladostigil, ungeremine, lactucopicrin, pharmaceutically acceptable salts thereof, pharmaceutically acceptable solvates thereof and combinations thereof. In some embodiments, the acetylcholinesterase inhibitor is an organophosphate. In some embodiments, the acetylcholinesterase inhibitor is selected from diisopropyl fluorophosphates, phospholine iodide (echothiophate), parathion, malathion, diazinon, pharmaceutically acceptable salts thereof, pharmaceutically acceptable solvates thereof and combinations thereof. In some embodiments, the cannabanoid is selected from Δ9-tetrahydrocannabinol, a synthetic cannabinoid, a semisynthetic cannabinoid and combinations thereof. In some embodiments, the neuroprotective agent is selected from acetyl-L-carnitine, xaliproden, cerebrolysin, and combinations thereof. In some embodiments, the muscarinic receptor modulator is selected from sabcomeline, milameline, xanomeline, oxotremorine, pharmaceutically acceptable salts thereof, pharmaceutically acceptable solvates thereof, and combinations thereof. In some embodiments, the prostanoid receptor agonist is selected from prostaglandin E1, limaprost, gemeprost, mesoprostol, prostaglandin E2, dinoprostone, sulprostone, prostaglandin F₂ₐ, carboprost, and combinations thereof. In some embodiments, the inhibitory oligonucleotide molecule is selected from a dsRNA, siRNA, shRNA, miRNA, piRNA, external guide sequences, ribozymes, short catalytic RNAs, and combinations thereof. In some embodiments, the inhibitory oligonucleotide molecule comprises an antisense oligodeoxynucleotide less than 30 nucleotides in length. In some embodiments, the inhibitory oligonucleotide molecule has a base sequence substantially complementary to a sequence of a target mRNA. In some embodiments, the inhibitory oligonucleotide molecule has a base sequence substantially complementary to a sequence of a target mRNA precursor. In some embodiments, the target mRNA is mRNA complementary to a gene encoding an acetylcholinesterase protein. In some embodiments, the inhibitory oligonucleotide molecule comprises one or more of the following: a phosphodiester backbone, a phosphorothioate backbone, a locked nucleic acid, peptide nucleic acid, tricycle-DNA, a decoy oligonucleotide, a ribozyme, a spiegelmer, a CpG oligomer, and combinations thereof. In some embodiments, the lipid vehicle comprises a liposome of cationic and non-ionic lipids. In some embodiments, the lipid vehicle to inhibitory oligonucleotide molecule molar ratio ranges from at least about 1:1 to about 20:1. In some embodiments, the composition is locally administered by a route selected from instillation, drug eluting matrix, drug eluting device, topical, and combinations thereof.

In some embodiments, the composition may further include a pharmaceutically and/or physiologically acceptable carrier, excipient, and/or diluent. In some embodiments, the composition is locally administered by a route selected from instillation, drug eluting matrix, drug eluting device, topical, and/or combinations thereof.

The present invention is, at least in part, directed to pharmaceutical composition for local administration to the bladder which include, but are not limited to, solutions, powder, fluid emulsions, fluid suspensions, semi-solids, ointments, pastes, creams, gels and jellies, and foams. Active ingredients in such compositions can be contained with pharmaceutically acceptable diluents, hydrophobic vehicles, water soluble vehicles, fillers, disintegrants, binders, lubricants, surfactants, emulsifiers, buffers, humectants, moisturizers, solubilizers, preservatives and the like. Means and methods of administration are known in the art, e.g. Goodman & Gilman's The Pharmaceutical Basis of Therapeutics, 6^{th} Edition, MacMillan Publishing Co. New York (1980) which is incorporated for all purposes. In some embodiments, local application is to urothelial tissue.

Examples of pharmaceutically acceptable vehicle include, but are not limited to, lipid vehicles, degradable or non-degradable polymers, hydrogels, cyclic oligosaccharides, surfactant, virosomes, and the like.

In some embodiments, the lipid vehicle may be a liposome. The lipid vesicles may comprise either one or several aqueous compartments delineated by either one (unilamellar) or several (multi-lamellar) lipid bilayers. In some embodiments, the liposomes may include cationic lipids and in yet other embodiments, the lipids may include non-ionic lipids. Lipid vehicles include, but are not limited to, phospholipids, glycolipids, sphingolipids, sphingophospholipids, or sphingoglycolipids. While not being bound by theory, liposomes may be adsorbed, or fuse, or transfer lipids with the cell membrane, and they can be endocytosed inside the cell.

The phospholipid vehicles may be any natural or synthetic phospholipid including, but not limited to, phosphatidylcholine, phosphatidic acid, phosphatidylethanolamine, phosphatidylserine, phosphatidylinositide, phosphatidylglycerol, phosphosphingolipids, cardiolipin, sphingomyelin, sphingosine I-phosphate, ceramidegalactopyranoside, ganglioside, cerebroside, cholesterol, 1,2-distearoylsn-glycero-3-phosphocholine, or 1,2-dioleoylphosphatidylcholine.

The glycolipid vehicles include, but are not limited to, galactolipids, sulfolipids, glycosphingolipids, cerebrosides, galactocerebrosides, glucocerebrosides, sulfatides, gangliosides, globosides, glycophosphosphingolipids, or glycosylphosphatidylinositols.

In some embodiments, the pharmaceutically acceptable vehicle may be a polymer or an organic macromolecule such as, for example, hydrophilic polymers (polyethylene oxide, polyoxyethylene-polyoxypropylene copolymers, polyvinyl alcohols, and the like), cellulosic polymers (hydroxypropyl cellulose, hydroxyethyl cellulose, hydroxypropyl methylcellulose, hydroxypropyl methylcellulose, phthalate, methyl cellulose, and the like), tragacanth or xanthan gums, sodium alginate, and the like, or combinations thereof.

In some embodiments, the pharmaceutically acceptable vehicle may be a surfactant such as, for example, lecithin, hydrogenated lecithin, naturally occurring lecithin, egg lecithin, hydrogenated egg lecithin, soy lecithin, hydrogenated soy lecithin, and vegetable lecithin, poloxamers, polyoxypropylenepolyoxethylene block copolymers, pluronic surfactants (e.g. 1,2-Propyleneglycol, Adeka 25R1, Adeka 25R2, Adeka L 61, Adeka Pluronic F 108, AIDS 162017, AIDS-162017, Antarox 17R4, Antarox 25R2, Antarox B 25, Antarox F 108, Antarox F 68, Antarox F 88, Antarox F 88FL, Antarox L 61, Antarox L 72, Antarox P 104, Antarox P 84, Antarox SC 138, Area Polyol R 2633, Areal E 351, B 053, BASF-L 101, Berol TVM 370, Bloat guard, Block polyethylene-polypropylene glycol, Block polyoxyethylene-polyoxypropylene, Breox BL 19-10, BSP 5000, C13430, Cirrasol ALN-WS, Crisvon Assistor SD 14, CRL 1005, CRL 1605, CRL 8131, CRL 8142, D 500 (polyglycol), Daltocel F 460, Dehypon KE 3557, Detalan, Eban 710, Emkalyx EP 64, Emkalyx L 101, Emkalyx L101, Empilan P 7068, Emulgen PP 230, Epan 450, Epan 485, Epan 710, Epan 750, Epan 785, Epan U 108, Epon 420, Ethylene glycol-propylene glycol block copolymer, Ethylene glycol-propylene glycol polymer, Ethylene oxide-propylene oxide block polymer, Ethylene oxide-propylene oxide block copolymer dipropylene glycol ether, Ethylene oxide-propylene oxide block copolymer ether with ethylene glycol, Ethylene oxide-propylene oxide block polymer, Ethylene oxide-propylene oxide copolymer, F 108, F 127, F 77, F 87, F 88, F-108, Genapol PF 10, Polyethylenepolypropylene Glycols, Polyethylene-polypropylene Glycols, HSDB 7222, Hydrowet, Laprol 1502, LG 56, Lutrol F, Lutrol F (TN), M 90/20, Magcyl, Meroxapol 105, Methyloxirane polymer with oxirane block, Methyloxirane-oxirane copolymer, Methyloxirane-oxirane polymer, Monolan 8000E80, Monolan PB, N 480, Newpol PE-88, Max 1646, Max LG 56, Nissan Pronon 201, Nixolen SL 19, NSC 63908, NSC63908, Oligoether L-1502-2-30, P 103, P 104, P 105, P 123, P 65, P 84, P 85, PEG/PPG-125/30 Copolymer, Plonon 201, Plonon 204, Pluracare, Pluracol V, Pluriol PE, Pluriol PE 6810, Pluronic, Pluronic 10R8, Pluronic 3182, Pluronic C 121, Pluronic F, Pluronic F 108, Pluronic F 125, Pluronic F 127, Pluronic F 38, Pluronic F 68, Pluronic F 68LF, Pluronic F 87, Pluronic F 88, Pluronic F 98, Pluronic F108, Pluronic F127, Pluronic F68, Pluronic F-68, Pluronic F77, Pluronic F86, Pluronic 87, Pluronic F87-A7850, Pluronic F88, Pluronic L, Pluronic L 101, Pluronic L 121, Pluronic L 122, Pluronic L 24, Pluronic L 31, Pluronic L 35, Pluronic L 44, Pluronic L 61, Pluronic L 62, Pluronic L 64, Pluronic L 68, Pluronic L 92, Pluronic L-101, Pluronic 144, Pluronic L62, Pluronic 162 (mw 2500), Pluronic L64, Pluronic 164 (mw 2900), Pluronic L-81, Pluronic P, Pluronic P 104, Pluronic P 75, Pluronic P 85, Pluronic P103, Pluronic P104, Pluronic P105, Pluronic P123, Pluronic P65, Pluronic P-65, Pluronic P-75, Pluronic P84, Pluronic P85, Pluronic-68, Poloxalene, Poloxalene [USAMBAMINN], Poloxalene L64, Poloxalkol, Poloxamer, Poloxamer [USAN: BAMINN], Poloxamer 101, Poloxamer 108, Poloxamer 182LF, Poloxamer 188, Poloxamer 331, Poloxamer 407, Poloxamer-188, Poly (propylene oxide-ethylene oxide), Poly(ethylene oxide-co-propylene oxide), Poly(mixed ethylene and propylene) glycol, Poly(oxyethylene)-poly(oxypropylene) glycol, Poly(oxyethylene)-poly(oxypropylene) polymer, Polyethylene glycol, propoxylated, Polyethylene oxide-polypropylene oxide, Polyethylene oxide-polypropylene oxide copolymer, Polyethylene-Pluronic L-62LF, Polyethylene-polypropylene glycol, Polykol, Polylon 13-5, Polyoxamer 108, Polyoxyethylenated poly (oxy propylene), Polyoxyethylene-polyoxypropylene block copolymer, Polyoxyethylene-polyoxypropylene copolymer, Polyoxyethylene polyoxypropylene, Polyoxyethylene-oxy-propylene [French], Polyoxyethylene-polyoxypropylene, Polyoxyethylene-polyoxypropylene polymer, Polyoxypropylene-polyoxyethylene block copolymer, Polypropoxylated, polyethoxylated propylene glycol, Polypropylene glycol, Polypropylene glycol-ethylene oxide copolymer, PPGDial 3000EO, Proksanol, Pronon, Pronon 102, Pronon 104, Pronon 201, Pronon 204, Pronon 208, Propane-1,2-diol, ethoxylated propoxylated, Propylen M 12, Propylene glycol-propylene oxide-ethylene oxide polymer, Propylene oxide-ethylene oxide copolymer, Propylene oxideethylene oxide polymer, Proxanol, Proxanol 158, Proxanol 228, Proxanol Ts1-3, RC 102, Regulaid, Rokopol 16P, Rokopol 30P, Rokopol 30P9, SK and F 18,667, Slovanik, Slovanik 630, Slovanik 660, Slovanik M-640, Supronic B 75, Supronic E 400, Synperonic PE 30/40, Tergitol monionic XH, Tergitol nonionic XH, Tergitol XH, Tergitol XH (nonionic), Teric PE 61, Teric PE 62, Teric PE40, Teric PE60, Teric PE70, Thanal E 4003, Therabloat, TsL 431, TVM 370, Unilube 50MB168X, Unilube 50MB26X, Velvetol OE 2NT1, Varanol P 2001, WS 661, Wyandotte 7135, a-Hydro-Q-hydroxypoly(oxyethylene)ₐ-poly(oxopropylene)b-poly(oxyethylene)b block copolymer, and the like) and combinations thereof.

The pharmaceutically acceptable vehicle may, in certain embodiments, further include solvents such as, for example solvent ethyl laureate, ethyl myristate, isopropyl myristate, isopropyl palmitate, cyclopentane, cyclooctane, trans-decalin, trans-pinane, n-pentane, n-hexane, n-hexadecane, and tripropylamine; and the at least one polar agent is selected from water, alcohols, polyalcohols, glycerol, glycerols, polyglycerols, ethylene glycol, polyglycols, and formamide.

Pharmaceutical compositions of the therapeutic agents can also comprise suitable solid or gel phase carriers or excipients. Examples of such carriers or excipients include, but are not limited to, calcium carbonate, calcium phosphate, various sugars, starches, cellulose derivatives, gelatin, and polymers such as, for example, polyethylene glycols.

The compositions of the present invention can also be administered in combination with other active ingredients, such as, for example, adjuvants, protease inhibitors, or other compatible drugs or compounds where such combination is seen to be desirable or advantageous in achieving the desired effects of the methods described herein.

In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments, the diluent component comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the optional lubricant component, when present, comprises one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

This invention and embodiments illustrating the method and materials used may be further understood by reference to the following non-limiting examples.

### EXAMPLES

UAB can be induced in animals by several means, either by functional urethral constriction model or bilateral pelvic nerve crush method. The urinary bladder is innervated by the pelvic plexus. Parasympathetic postganglionic neurons that provide an excitatory input to the pelvic organs are located within the plexus. Bilateral pelvic nerve crush (PNC) involves clamping the preganglionic pelvic near the major pelvic ganglion (MPG) using clamp to crush each pelvic nerve for 60 seconds at site proximal to each pelvic nerve's entry into the major pelvic ganglion. PNC creates an acute model of UAB that can allows for testing of candidate agents. This was evaluated in female SD rats with additional animals having undergone sham pelvic nerve crush. By 4 weeks, no differences were seen in these measures suggesting recovery of bladder function from PNC.

Drug formulation of irreversible long-lasting organophosphorus compound echothiophate efficacy in UAB model is assessed.

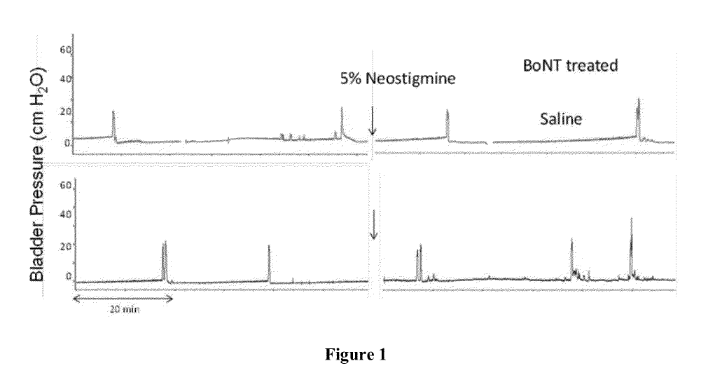

Prior studies have shown that systemic administration of AChE inhibitor increased the maximum bladder contraction pressure and duration of contraction without increasing the bladder baseline pressure. In our study a similar outcome through intravesical administration of neostigmine was achieved. The open transurethral CMG in female rats was conducted under urethane anesthesia (1.2 g/kg subcutaneous dose). 5% w/v concentration of neostigmine in saline was infused in rats previously treated by instillation of saline or botulinum toxin (BoNT). Instillation of neostigmine caused an increase in duration of voiding contraction, which is an accepted index of efficacy for agents improving UAB. Instillation of neostigmine in rats pretreated with saline also caused a decrease in the interval between bladder contractions as evident from reduced intracontractile interval between voiding peaks. (FIG. 1).

Preliminary data revealed that CMG of normal adult female rats pretreated with BoNT following neostigmine instillation do not show increased detrusor contractility during the filling phase. BoNT treatment by its characteristic negative effect on detrusor contractility partially simulates the condition of UAB in normal rats and infusion of neostigmine in such a condition improves contractility without affecting baseline pressure. Although slightly increased detrusor contractility during the filling phase was noticed following neostigmine instillation in normal rats that were pretreated with saline. Effect of neostigmine during filling phase is indicated by small non-voiding peaks in saline treated rats, which was abolished by BoNT treatment (FIG. 1). Comparatively speaking, the local administration of neostigmine attenuates the deleterious effect of neostigmine observed from systemic administration. There is a consensus view in the field that an ideal treatment of underactive bladder would either decrease the urethral resistance and/or the increase of bladder contraction pressure. These effects translate into increased the voided volume and the maximum flow rate without affecting either the intravesical pressure or the bladder compliance.

Method: The primary goal of the formulation optimization studies is to achieve drug loading conditions that would allow efficient loading of echothiophate iodide into liposomes. The rate of drug release from the liposomes will be manipulated by altering the liposomal lipid constituents and liposome particle size. Liposomes can thus be designed to achieve a sustained and predictable rate of drug delivery. Liposomes have been used to circumvent the problem of toxic side effects that may occur with bolus drug administration. Different methods will be tried for obtaining desired encapsulating efficiency of echothiophate iodide into liposomes (vesicles). The main requirements of an encapsulation method are that it facilitates high yield entrapment and stable encapsulation, easy to scale-up and the phospholipid composition confers a high stability to the liposome preparation. Various methods include, but are not limited to dehydration-rehydration and reverse-phase evaporation methods. Formulation optimization will be carried out using a 2×5 full factorial design based on five independent variables. The effects of the studied parameters on entrapment efficiency, particle size, and percentage of drug released after 1 and 5 h will be investigated. Encapsulation process will involve lyophilizing a mixture of echothiophate iodide, phospholipids, water and TBA. Stability of freeze-dried cakes in the lyophilization process is influenced by several technological factors: freezing protocols, drying protocols and extrinsic factors of storage conditions. The retention of drug inside liposomes, gel-to-liquid crystalline phase transition temperature T(m) and glass transition temperature T(g) will be selected as indicators to investigate the protective effect during lyophilization. Particle size of empty and loaded liposomes will be determined by dynamic light scattering or quasi-elastic light scattering (QELS) and electron microscopy (EM). The loaded liposomes carrying drug in the concentration 2, 4, 8 mg/ml will be stored at 4° C. until characterized further or used for in vivo studies.

Encapsulation efficiency: Drug encapsulation efficiency will be determined by the mini column centrifugation method. Disposable syringes (1 mL) will be first plugged with cotton and packed with hydrated Sephadex G-25M gel (1%, m/V). Sephadex G-25 will be previously soaked in 0.9% (V/V) saline for 4 h. These syringes will be placed in plastic centrifugal tubes and the whole assembly will be centrifuged at 6700′g for 15 min to make the bed dry. To this dried bed, 500 μL of liposomal suspension of echothiophate iodide will be added and the assembly centrifuged at 600×g for 15 min. The encapsulated drug will be separated based on the molecular size. This process will be repeated three times using fresh syringes packed with gel each time to ensure complete removal of non-entrapped free drug. The concentration of the encapsulated drug will be estimated by the HPLC method. The Waters HPLC system had C8 stationary phase (250 ‘4.6 mm, 5 mm) with a mobile phase composed of 20 mmol/L phosphate buffer pH 3.0 and acetonitrile (75:25%, V/V) at a flow rate of 1 mL/min. The. The detection wavelength will be 210 nm.

Expected Results: Liposomes with incorporated echothiophate iodide will provide sustained drug-release and enhance drug efficacy. Drug loaded liposomes will be more electron-dense than empty liposome on electron microscopy. A quantitative increase in pixel density/darkness is expected to be observed for the loaded liposomes. The mean particle size of loaded liposomes will be measured by the two methods of EM and QELS. QELS measures time-dependent fluctuations in scattered light intensity that result from particle diffusion, allowing for calculation of hydrodynamic size of the vesicles, which includes surface-bound water layers. In contrast, electron microscopy measures only the diameter of the lipid membrane and also allows discrimination of larger, non-liposomal particles that can have a significant influence (“over-weighted”) in QELS measurements. Lyophilization is a promising approach to ensure the long-term stability of liposomes. The LP-08 lipid is not only biocompatible but also imparts a high degree of membrane stability (reduced leakage), due to factors including suppression of the gel-to-liquid crystalline phase transition.

Contractility Studies: The ability of lipo-echothiophate to enhance endogenous acetylcholine-induced detrusor smooth muscle contractions will be assessed in vitro using isometric tension measurements. A day after instillation of lipo-echothiophate, the rats will be euthanized by decapitation and bladder strips of approximately 20 mg will be prepared. The preparations will be mounted in 5 ml organ baths containing Krebs and constantly bubbled with a mixture of 95% 02 and 5% CO2. The initial tension will be set to 10 mN and isometric contractions were measured with strain-gauge transducers (Grass F3) coupled to a TBM4 strain gauge amplifier and recorded on a computer-based data acquisition program (Powerlab). The sampling rate per channel will be set to 100 Hz. Field stimulation will be used to study nerve-mediated activity. After 40-min equilibration, electrical field stimulation was applied through two platinum wire electrodes positioned on the top and the bottom of the organ bath separated by 4 cm. The bladder strips will be stimulated with square wave pulses of 0.25 ms duration with a voltage setting that produces maximal contraction amplitude (20 V/cm). Frequency-response curves will be constructed by stimulating bladder strips at various frequencies (1-40 Hz) with trains of 10 and 80 shocks. The contraction parameters will be normalized on the basis of tissue cross-sectional area calculated from the weight, resting length, and density (1.05 g/cm³) of the bladder tissue and will be expressed as mN/mm². The amplitude and maximal rate of the contractile responses were computed by a calculation program in Powerlab. Strength-duration curves in the presence of tetrodotoxin (10⁻⁷ g/ml) will be constructed.

Bilateral pelvic nerve crush model: The procedure will involve exposing the pelvic nerves bilaterally through a lower midline abdominal incision under isoflurane anesthesiaas they enter into the major pelvic ganglion. The bladder will be lifted laterally to expose the MPG better as it lies just lateral to the urethra near the bladder neck. A straight micro mosquito clamp will be used to crush each pelvic nerve in the experimental group for 60 seconds. For those rats in the control group the pelvic nerves bilaterally will be exposed but not crushed. Groups will be evaluated with isovolumetric cystometry at 1 week after bilateral pelvic nerve crush or sham operation. Animals testing will be randomly assigned in the groups and product tested by blinds. A day after PNC procedure total of 32 adult female Sprague-Dawley rats will be divided into four groups of 8 rats each to be instilled under isoflurane anesthesia for 30 min with either 1) PNC+empty liposome 2) PNC+Lipo-echothiophate 3) sham PNC+empty liposomes 4) sham PNC+Lipo-echothiophate. Animal will be allowed to recover from anesthesia with free access to food and water.

Metabolic Cage & Cystometric study: After treatments the animal will undergo 24 h metabolic cage evaluated of micturition. Baseline micturition pattern before PNC will be established for each rat. After metabolic cage study, in vivo terminal CMG will be done at 2, 4 and 7 days after Lipo-echothiophate treatment. Transurethral open CMG will be done on rats under urethane anesthesia (1.2 mg/kg s.c). Bladder infusion will be stopped at the end of each micturition and residual volume (RV) will be measured by manually compressing the bladder and measuring the expelled volume. Alternatively, cumulative volume of infusion subtracted from the carefully measured voided urine volume can provide PVR. Bladder capacity (BC) will be calculated as the sum of VV and PVR. Bladder compliance will be calculated in ml/cm water according to the formula, compliance=maximal bladder volume/opening pressure-baseline pressure (=[BC/(PT-BP)]). Based on these values, voiding efficiency (VE, %=[(VV/BC)×100]) could be estimated. ICI, maximal voiding pressure (MVP), pressure threshold for voiding (PT) and baseline pressure (BP) will also be measured.

Expected results: We expect to see a dose dependent augmentation of bladder contractility in response to field stimulation in bladder strip studies by lipo-echothiophate. Our previous studies showed that cholinergic component of the detrusor contractions is more prominent during longer train stimulation (80 shocks) than during shorter train (10 shocks) stimulation. Efficacy is expected at lower doses for liposomal echothiophate than for free echothiophate drug in saline solution. We do not expect any effect on micturition pattern in metabolic cage study at lower doses (in awake condition). Echothiophate may produce incontinence at higher doses and leaking of urine from urethral meatus. Incontinence will be confirmed by higher urinary frequency. We expect lipo-echothiophate treated PNC rats to show reduced PVR compared to PNC rats treated with empty liposomes. Groups 3 and 4 with sham PNC injury will serve as a guide in interpreting the experimental results.

Liposomes Restrict Systemic Uptake of Instilled Radioactivity: Radioactive lipid was included in small mole fractions (1.64% w/w) relative to total lipids used for making liposomes. Total lipid dose (1 mg) for each animal was dispersed in 0.5 ml of saline. Given the specific activity of lipid to be 52 mCi/mmol, each animal received a radioactive dose of 0.97|iCi or −81000 disintegrations per minute (dpm). Rats were kept in metabolic cages after liposome instillation and urine was collected to measure radioactive counts. The majority of the liposome was recovered in the urine with cumulative excretion of up to 35.51±7.12% of instilled dose for the time period 0-3 h after instillation. Given the slow elimination rate constant, we estimated the bladder residence time of liposome of 24 h after instillation that was also confirmed by bladder tissue levels of radioactivity. Analysis of bladder tissue showed that 0.06±0.01% of dose was absorbed into the bladder by 3 h, indicating that liposomes restrict the action of instilled drugs locally in the bladder and avoid systemic side effects.

Animal Model of Underactive Bladder: UAB can be induced in animals by several means, either by functional urethral constriction model or bilateral pelvic nerve crush method. Bilateral pelvic nerve crush (PNC) involves clamping the preganglionic pelvic nerve near the major pelvic ganglion (MPG) for 60 seconds. PNC creates an acute model of UAB that can allow us testing of candidate agents in the Phase I of this SBIR project. In Phase II, we will institute chronic models of UAB including aging and diabetic rats.

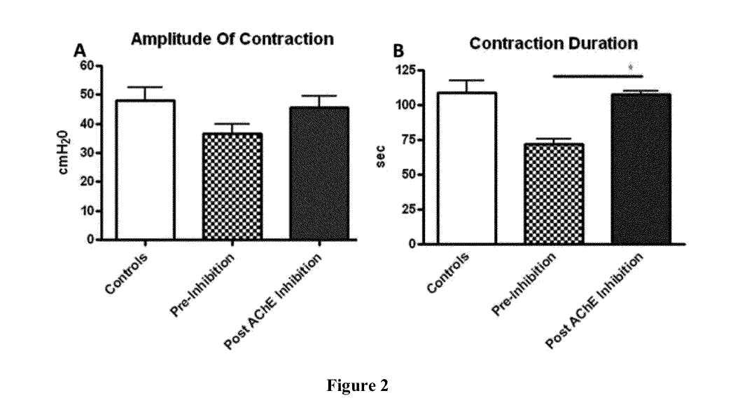

Intravesical AChE inhibitor infusion: We have proof of concept studies with intravesical administration of neostigmine using open transurethral CMG under urethane anesthesia (1.2 g/kg,s.c). The cystometry of control untreated rats was compared with rats treated with botulinum toxin (BoNT) 5 days earlier and then infused with 149.5 mM Neostigmine on day of cystometry after saline infusion at rate of 0.04 ml/min. BoNT treatment has a characteristic negative effect on detrusor contractility, which partially mimics the condition of UAB in normal rats as reflected by the bar representing pre-inhibition condition (before infusion of neostigmine during CMG) in FIG. 2. Instillation of neostigmine caused a significant increase in duration of contraction (*ANOVA followed by Tukey multiple comparison test). The pilot data support our hypothesis that duration of detrusor contraction is dependent upon reflex efferent nerve activity in response to increased sensory activity induced by prolonged activity of acetylcholine. Neostigmine infusion also raised amplitude of contraction relative to pre-infusion stage (pre-inhibition). Proposed experiment will advance this hypothesis further by testing effect of reduced expression of AChE in bladder after siRNA treatment in pelvic nerve crush model of UAB.

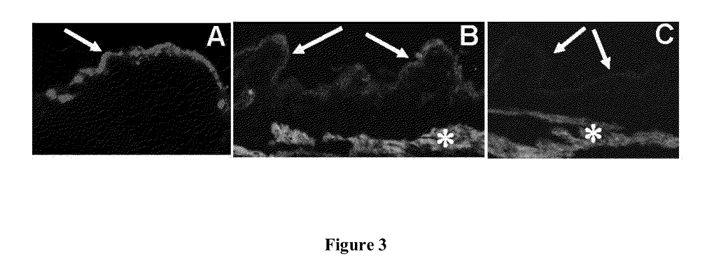

Strategy of Liposomal Bladder Delivery Platform: Intravesical drug delivery has had success and unique advantages. Three compounds developed include delivery BoNT for refractory overactive bladder, tacrolimus for hemorrhagic cystitis, and antisense for nerve growth factor (NGF) potentially for neurogenic bladder dysfunction. Intravesical application of NGF antisense-liposome conjugates results indicate that antisense inhibitory oligonucleotide conjugated with liposomes is effectively taken into the urothelial layer following intravesical delivery FIG. 3 (A), and that NGF antisense-liposome conjugates can decrease NGF expression in the urothelium, but not the detrusor FIGS. 3 (B & C).

Cationic liposomes for the experiments, composed of DOTAP (N-[1-(2,3-Dioleoyloxy)propyl]-N,N,Ntrimethylammonium Methylsulfate), were made by a thin film hydration method and hydrated with nuclease free water with the final lipid concentration of 7 mM. Oligos with the sequence 5′G*C*C*CGAGACGCCTCC*C*G*A 3′ (SEQ ID NO:3) were dispersed in nuclease free water at the concentration of 6 mM and were complexed with liposomes by incubation at room temperature for 30 min.

FIG. 3 (A) shows the confocal photomicrograph of the bladder 24 hrs after instillation of NGF antisense oligos tagged with a red fluorescent dye (TYE563) conjugated with LP to demonstrate bladder UT uptake of fluorescent labeled oligos An arrow indicates the urothelial layer. (Magnification 200×). FIGS. 3 (B & C) illustrate the effect of delivered antisense inhibitory oligonucleotide on NGF expression in the urothelium was studied by green (Alexa 488) staining for NGF immunoreactivity. Inhibitory oligonucleotide delivered by liposomes (panel C) reduced green NGF immunoreactivity in urothelium compared to the control rat treated with saline (panel B) (Magnification 200×). Arrows indicate the urothelial layer. Note that green fluorescence is shown in the saline treated bladder urothelium (B) while fluorescent signals are much reduced in anti-NGF oligo-LP-treated bladder urothelium (C). There are no changes in NGF immunoreactivity of the detrusor muscle layer (*) between B and C.

The siRNA molecule is polyanionic and as such it cannot passively diffuse across most cell membranes including glycoaminoglycan layer (anionic charge) lining the bladder lumen. Preliminary experiments were done to determine the intravesical uptake of liposomes complexed with oligonucleotide, which can be considered surrogate for siRNA. The fluorescence uptake studies demonstrate influx of liposomal antisense oligo from single instillation and we expect to refine our formulation further and optimize the dose regimen for effective suppression of AChE expression in urothelium in proposed studies.

Experimental/Research Design and Methods: Aim 1: To formulate siRNA against AChE into liposomes for intravesical delivery.

Method: We plan to use the published sequences for sense and antisense oligonucleotides for siRNA targeting AChE: 5′-AAAAGGTGGTAGCATCCAATACCTGTCTC-3′ (SEQ ID NO:4) and 5′-AATATTGGATGCTACCACCTTCCTGTCTC-3′ (SEQ ID NO:5). The siRNA macromolecule is vulnerable to rapid hydrolysis by extracellular nucleases present in urine and complexation with liposomes can protect against nucleases. Lyophilized siRNA of ultra-high purity r cationic lipid, 1,2-dioleoyl-3-trimethylammonium-propane. This reconstituted mixture will be vortexed for 2 minutes, allowed to hydrate at room temperature over 0.5 h. The entrapment efficiency, particle size, and percentage of siRNA released at different time points will be investigated. Particle size of empty and loaded liposomes will be determined by dynamic light scattering and electron microscopy (EM). The loaded liposomes carrying siRNA will be stored at 4° C. until characterized further or used for in vivo studies.

Trapping efficiency of siRNA in liposomes will be calculated using Texas Red-labeled siRNA. Liposomes complexed with labeled siRNA will be dissolved in the same amount of lysis buffer (2 mM EDTA and 0.05% Triton-100 in pH 7.8 Tris buffer). The standard siRNA solutions will be prepared by diluting the Texas Red-labeled siRNA in the same lysis buffer. Then, 100 μl of each standard and liposome complex will be taken to 96-well plate and the fluorescence intensity will be measured on plate reader to record the fluorescence intensity at 615 nm. The amount of entrapped siRNA will be calculated according to the standard calibration curve.

Expected Results: Previous studies on use of intravesical liposomes using a test drug have shown that liposomes significantly increase the area under the curve of bladder exposure of drugs compared to saline solution of test drug We expect that: (1) the formulation optimization studies will achieve conditions that would allow efficient complexing of siRNA into liposomes. Lyophilization is a promising approach to ensure the long-term stability of liposomes. The lipid is not only biocompatible but also imparts a high degree of membrane stability due to factors including suppression of the gel-to-liquid crystalline phase transition.

Aim 2: Demonstrate success and in vivo efficacy and safety of siRNA against AChE in rat model of induced UAB.

Methods: Efficacy: 12 groups of 3 female SD rats of 200-250 grams (n=36) at each dose level will be studied. Rats will be instilled with either saline, liposome complexed with siRNA at concentrations of 1, 5, 10, 20 micromolar, respectively. Another set of rat groups will be given naked siRNA at concentrations of 1, 5, 10, 20 micromolar, respectively. Bladder instillation will be done under isoflurane anesthesia for 30 min and animal will be allowed to recover from anesthesia with free access to food and water.

Contractility Studies: The ability of lipo-siRNA to enhance endogenous acetylcholine-induced detrusor smooth muscle contractions through reduced expression of AChE will be assessed in vitro using isometric tension measurements as previously described by our group (Somogyi et al. 2002). In brief, the tissue obtained from bladder body above the urethral orifices will longitudinally be cut into strips (approximately 0.5×0.5×7 mm in size). Electrical field stimulation (EFS) will be used to study nerve-mediated activity. During EFS, the intrinsic nerves will be stimulated with rectangular pulses of 5 msec duration and 20 V, at frequencies of 0.5-64 Hz. Trains of pulses will last for 2 sec and the stimulation interval will be 120 sec. After control levels of EFS-induced contractility are recorded, EFS will be repeated in the presence of atropine (10-6 g/ml) to assess the level of acetylcholine-dependent contraction in EFS-induced contractions. The contraction parameters will be normalized on the basis of tissue cross-sectional area calculated from the weight, resting length, and density (1.05 g/cm3) of the bladder tissue and will be expressed as mN/mm2. Strength-duration curves in the presence of tetrodotoxin (10-7 g/ml) will also be constructed to confirm that detrusor contraction during EFS is mediated by nerve activation.

Bilateral pelvic nerve crush model: The procedure will involve exposing the pelvic nerves bilaterally through a lower midline abdominal incision under isoflurane anaesthesia as they enter the MPG shown in FIG. 2. The bladder will be lifted laterally to expose the MPG better as it lies just lateral to the urethra near the bladder neck for 60 seconds. Pelvic nerves bilaterally will be exposed but not crushed in controls. Animals testing will be randomly assigned in the groups and product tested blinded. A day after the PNC procedure, a total of 32 adult female SD rats will be divided into four groups of 8 rats each to be instilled under isoflurane anesthesia for 30 min with either 1) PNC+empty liposome 2) PNC+Lipo-siRNA 3) sham PNC+empty liposomes 4) sham PNC+Lipo-siRNA. Animal will be allowed to recover from anesthesia with free access to food and water. Groups will be evaluated with isovolumetric cystometry at 1 week after PNC or sham operation.

Metabolic Cage & Cystometric study: After treatments the animal will undergo 24 h metabolic cage evaluated of micturition. Baseline micturition pattern before PNC will be established for each rat. After metabolic cage study, in vivo terminal CMG will be done 7 days after Lipo-siRNA treatment under urethane anesthesia (1.2 g/kg s.c). Bladder infusion will be stopped at the end of each micturition to measure residual volume (RV) by manually compressing the bladder and measuring the expelled volume. Bladder capacity (BC) will be calculated as the sum of voided volume (VV) and RV. Bladder compliance will be calculated in ml/cm of water=maximal bladder volume/opening pressure−baseline pressure (=[BC/(PT−BP)]). Based on these values, voiding efficiency (VE, %=[(VV/BC)×100]) will be estimated.

Expected results: We expect to see a dose dependent augmentation of atropine-sensitive (=ACh-dependent) bladder contractility in response to field stimulation in bladder strip studies by lipo-siRNA. We expect to show the need for liposomes in intravesical delivery of siRNA. Lipo-siRNA may produce incontinence at higher doses and leaking of urine from urethral meatus. Incontinence will be confirmed by overflow incontinence on CMG. We expect lipo-siRNA treated PNC rats to show reduced PVR compared to PNC rats treated with empty liposomes. Groups 3 and 4 with sham PNC injury will serve as a guide in interpreting the experimental results.

Pitfalls and remedies: Post-void residual is important outcome for these experiments and it can also be measured by subtracting carefully measured voided urine volume from cumulative volume of infusion. To confirm a dose dependent inhibition of AChE activity by siRNA, we will perform assay for AChE activity by Ellman method that can also assess effective delivery of siRNA to rat bladder by liposomes.

Detailed Experimental Methods:

Bladder instillation: The lipo-siRNA will be instilled into the bladder using a 24-gauge Teflon catheter under isoflurane anaesthesia and withdrawn at end of instillation without exposing the bladder contents to urethra.

Cystometry: A PE-50 polyethylene tube will be inserted transurethral and connected via a 3-way stopcock to a pressure transducer and to a syringe pump for recording intravesical pressure and infusing saline into the bladder at a rate of 0.04 ml/min to elicit repetitive bladder contractions. Cystometric parameters will be measured during saline infusion and saline voided from the urethral meatus will be collected and measured to determine voided volume (VV). Data will be stored using data acquisition software (AD Instruments, Australia).

Micturition Patterns: Urination pattern profiles will be obtained in rats using specialized metabolic cages (Nalgene, Rochester, N.Y.) and parameters evaluated in this study will include total urine output/24 h, number of micturitions/24 h, and mean volume per micturition (Ozawa et al. 1999).

Data analysis: In quantitative experiments, the parametric tests (Student t test, one way ANOVA with Dunett or Newman-Keuls test) using GraphPad at level of p<0.05. Repeated-measures ANOVAs with least significant difference post hoc tests will be performed between all groups to determine statistical significance across time and between groups.

Although the present invention has been described in considerable detail with reference to certain preferred embodiments thereof, other versions are possible. Therefore, the spirit and scope of the appended claims should not be limited to the description and the preferred versions contained within this specification.

What is claimed is:

1. A method for treating an underactive bladder condition or symptoms thereof comprising: locally administering to the bladder a composition comprising a pharmaceutically acceptable lipid vehicle and an effective amount of a therapeutic agent, wherein the underactive bladder condition or symptoms thereof are treated.

2. The method of claim 1, wherein the underactive bladder condition or symptoms thereof is selected from urinary retention, incomplete bladder emptying, slow or intermittent stream, hesitancy, terminal dribbling, straining to urinate that is related to diabetes, neurological diseases affecting the nerves to and from the bladder, bladder outlet obstruction and impaired contractility of detrusor muscle and combinations thereof.

3. The method of claim 1, wherein the underactive bladder condition or symptoms thereof is caused by a condition selected from benign prostate hyperplasia, urethral stricture, paruresis, detrusor muscle underactivity, underactive bladder syndrome, anticholinergic drug effect, bladder sensory dysfunction, diabetes and combinations thereof.

4. The method of claim 1, wherein the effective amount of the therapeutic agent is an amount less than the amount administered systemically to obtain the same therapeutic efficacy.

5. The method of claim 1, wherein the local administration is selected from instillation, drug eluting matrix, drug eluting device, topical and combinations thereof.

6. The method of claim 1, wherein the composition further comprises a physiologically acceptable carrier, excipient or diluent.

7. The method of claim 1, wherein the lipid vehicle comprises one or more of the following: a phospholipid, a glycolipid, a sphingolipid, a sphingophospholipid, a sphingoglycolipid, and combinations thereof.

8. The method of claim 1, wherein the lipid vehicle comprises one or more of the following: phosphatidylcholine, phosphatidylethanolamine, phosphatidylserine, phosphatidylinositol, phosphatidylglycerol, cardiolipin, sphingomyelin, sphingosine I-phosphate, ceramidegalactopyranoside, ganglioside, cerebroside, cholesterol, 1,2-distearoylsn-glycero-3-phosphocholine, 1,2-dioleoylphosphatidylcholine and combinations thereof.

9. The method of claim 1, wherein the therapeutic agent is selected from an acetylcholinesterase inhibitor, a potassium channel blocker, a cannabinoid, a muscarinic receptor agonist, a muscarinic receptor modulator, a neurotrophin, a ghrelin receptor agonist, a neuroprotective agent, a prostanoid receptor agonist, an inhibitory oligonucleotide molecule, and combinations thereof.

10. The method of claim 9, wherein the acetylcholinesterase inhibitor is a carbamate.

11. The method of claim 9, wherein the acetylcholinesterase inhibitor is selected from physostigmine, neostigmine, rivastigmine, pyridostigmine, ambenonium, demarcarium, tacrine, donepezil, distigmine, phenserine, galantamine, edrophonium, huperzine A, ladostigil, ungeremine, lactucopicrin, pharmaceutically acceptable salts thereof, pharmaceutically acceptable solvates thereof, and combinations thereof.

12. The method of claim 9, wherein the acetylcholinesterase inhibitor is an organophosphate.

13. The method of claim 9, wherein the acetylcholinesterase inhibitor selected from diisopropyl fluorophosphates, phospholine iodide (echothiophate), parathion, malathion, diazinon, pharmaceutically acceptable salts thereof, pharmaceutically acceptable solvates thereof, and combinations thereof.

14. The method of claim 9, wherein the cannabanoid is selected from Δ⁹-tetrahydrocannabinol, a synthetic cannabinoid, a semisynthetic cannabinoid and combinations thereof.

15. The method of claim 9, wherein the neuroprotective agent is selected from acetyl-L-carnitine, xaliproden, cerebrolysin, and combinations thereof.

16. The method of claim 9, wherein the muscarinic receptor modulator is selected from sabcomeline, milameline, xanomeline, oxotremorine, pharmaceutically acceptable salts thereof, pharmaceutically acceptable solvates thereof and combinations thereof.

17. The method of claim 9, wherein the prostanoid receptor agonist is selected from prostaglandin E1, limaprost, gemeprost, misotprostol, prostaglandin E2, dinoprostone, sulprostone, prostaglandin F₂ₐ, carboprost, and combinations thereof.

18. The method of claim 9, wherein the inhibitory oligonucleotide molecule is selected from a dsRNA, siRNA, shRNA, miRNA, piRNA, external guide sequences, ribozymes, short catalytic RNAs, and combinations thereof.

19. The method of claim 9, wherein the inhibitory oligonucleotide molecule comprises an antisense oligodeoxynucleotide less than 30 nucleotides in length.

20. The method of claim 9, wherein the inhibitory oligonucleotide molecule has a base sequence substantially complementary to a sequence of a target mRNA.

21. The method of claim 9, wherein the inhibitory oligonucleotide molecule has a base sequence substantially complementary to a sequence of a target mRNA precursor.

22. The method of claim 21, wherein the target mRNA is mRNA complementary to a gene encoding an acetylcholinesterase protein.

23. The method of claim 9, wherein the inhibitory oligonucleotide molecule comprises one or more of the following: a phosphodiester backbone, a phosphorothioate backbone, a locked nucleic acid, peptide nucleic acid, tricycle-DNA, a decoy oligonucleotide, a ribozyme, a spiegelmer, a CpG oligomer, and combinations thereof.

24. The method of claim 1, wherein the lipid vehicle comprises a liposome of cationic and non-ionic lipids.

25. The method of claim 1, wherein the lipid vehicle to inhibitory oligonucleotide molecule molar ratio ranges from at least about 1:1 to about 20:1.

26. The method of claim 1, wherein the composition is administered via intravesicular instillation.
